(19) United States
(12) Patent Application Publication
(10) Pub. No.: US 2022/0110545 A1
(43) Pub. Date: Apr. 14, 2022

(54) FALL DETECTOR SYSTEM AND METHOD

(71) Applicant: Healthcare Integrated Technologies Inc., Knoxville, TN (US)

(72) Inventors: Kenneth M. GREENWOOD, Davenport, FL (US); Scott Michael BORUFF, Knoxville, TN (US); Jurgen VOLLRATH, Sherwood, OR (US)

(21) Appl. No.: 17/500,914

(22) Filed: Oct. 13, 2021

Related U.S. Application Data
(60) Provisional application No. 63/204,621, filed on Oct. 14, 2020.

(51) Int. Cl. A61B 5/11; A61B 5/00

(52) U.S. Cl. CPC A61B 5/1117; A61B 5/746; A61B 5/1128

(57) ABSTRACT

A method and system for detecting a person falling, confirming a potential fall, and taking action to mitigate the effect.

### FIELD OF THE INVENTION

The invention relates to medical care. In particular it relates the detection of falls

### BACKGROUND OF THE INVENTION

People in old age homes, care facilities, hospitals, or who are living alone are at greater risk when it comes to falling. Not only are the elderly and infirm more likely to fall, but the consequences are also more severe, especially when there is no one around to provide help in a timeous manner.

Wearable fall detectors, like the FitBit wristwatch and Life Alert pendant have limited accuracy in identifying when a user has fallen. In many instances, the fall may be a gradual lowering to the ground as a user feels dizzy or is starting to pass out. In these situations, accelerometer-based devices are extremely unreliable in detecting falls. Also, insofar as a user has passed out, they may not be in a position to notify emergency response teams or family members to alert them that there is an emergency.

Studies have also shown that many users take their wearables off at night when they go to bed, thus leaving them exposed if they have to get up in the middle of the night or if they forget to put the wearable device back on again the next morning.

The present invention seeks to address these shortcomings.

### SUMMARY OF THE INVENTION

The present invention seeks to avoid the need for the user to have to wear or carry a fall-detection device.

According to one aspect of the invention, the fall detection system of the invention comprises a room monitoring device comprising a first detection device for capturing information about a user in a defined environment, and a second detection device, to validate information captured by the first device.

One or both of the first and second detection devices may be image capture devices. The two devices may comprise the same types of image capture devices, e.g., both radio frequency devices. They may also be of different types, selected, for example from, radio frequency, radar, sonar, lidar, visual spectrum video cameras, infra-red cameras.

The data captured by one image capture device may have limitations that can be supplemented with data from the other image capture device. For instance, the one image capture device may be a radar device that is more prone to interference by metal objects or otherwise more prone to defining false falls (false positives) or missing falls (false negatives). The other image capture device may be an infrared camera that shows a clearer picture of the user, thereby allowing a fall to be more accurately detected or verified. However, it may be more invasive into the user's privacy and therefore be implemented with black-out zones such as the bathroom.

The system may include a processor and memory configured with machine readable code defining an algorithm that controls the processor. One of the image capture devices may comprise a video camera, wherein the algorithm includes an object recognition algorithm to distinguish a person lying from a person standing, and for generating an alert if a person is detected lying down, other than on designated locations such as bed and sofa. The algorithm may also identify a person on the floor (lying or sitting), by distinguishing floor regions from bed or sofa regions.

Further, according to the invention, there is provided a method of enhancing the safety of a user in a dwelling, comprising monitoring the user for falls in the dwelling by capturing image data from at least two image capture sources, and corroborating the image data from the first source with data from the second source over a corresponding time frame in the event of a suspected fall.

The data not captured by the image capture device may include failures of the image capture device to identify a fall (false negative), designating something as fall when there was no fall (false positive), dead zones not covered by the image capture device, and regions designated as private areas.

In order to protect privacy, the image quality of the image capture device may have limitations inherent in the image capture device or built into the image capture device. For instance, the image capture device may include a radar or sonar device, or a camera operating in a frequency range outside the visual spectrum that inherently limits image quality, or a digital camera system where the pixel count is kept low enough to limit image quality to protect privacy.

According to another aspect of the invention, there is provided a system and method of monitoring patients, inmates, or other people requiring monitoring (generally referred to herein as the monitoree), which comprises generating a virtual doll-house of the monitoree's environment, generating a digital twin (also referred to herein for ease of reference as an avatar irrespective of the detail of the digital twin) of the monitoree, e.g. by parsing image data of the monitoree to define select body parts, generating a wire frame or other representation of the monitoree, synchronizing movement of the select body parts of the avatar according to movement of the corresponding select body parts of the monitoree, integrating the avatar into the environment, and monitoring the avatar for defined activities. The defined activities may include one or more of: fall events, interactions with other people, repetitive behavior, and impermissible behavior. This may include comparing the distance of one or more body parts relative to the floor.

Still further according to the invention, there is provided a fall detector, comprising at least two sensors for detecting falls, wherein one sensor detects a fall and the at least one other sensor corroborates the fall.

At least one of the sensors may be an image detector, or may comprise two image detectors of different types. One image detector may comprises a radar device and a second image detector may comprise an RGB or IR camera.

Thus, according to the invention there is provided a fall detector to detect falls by a person in a living space, comprising an image capture device, and a processor connected to a memory that includes machine-readable code defining an algorithm for analyzing data from the image capture device to detect falling events, wherein the image capture device is connected to a motion detector in order to capture only select information, and the algorithm includes logic to generate a digital twin of the person and monitor the distance of one or more body parts of the digital twin relative to the floor of the living space.

The algorithm may include logic to obfuscate at least part of the image, and may further include logic to define a flagging event if the distance of said one or more body parts of the digital twin relative to the floor drops below a defined value.

The algorithm may be configured to generate an alert to one or more persons when a flagging event is detected.

Further, according to the invention, there is provided a fall detector to detect falls by a person in a living space, comprising at least two image capture devices for detecting falls, a processor, a control memory connected to the processor and configured with machine-readable code defining an algorithm, and a data memory, wherein the algorithm includes logic to corroborate a fall detected by one image capture device by comparing the data from the other image capture device for a related time frame.

The image capture devices may operate in different frequency bands, e.g., one image detector may comprise a radar detector and a second image detector may comprise an RGB or IR camera.

The radar detector may be mounted in privacy-sensitive locations of the living space.

The algorithm may include logic to time the person in privacy-sensitive locations and identify anomalies in the timing and corroborate anomalies in the time spent by the person in the privacy-sensitive location with data from the radar detector for a related time frame.
For purposes of this application, the term algorithm includes one or more algorithms and may include the use of an artificial intelligence (AI) system to implement the logic of the algorithm.

Still further, according to the invention, there is provided a method for detecting a fall by a person in a living space, comprising monitoring the person in the living space by generating a digital twin of the person, monitoring one or more body parts of the digital twin relative to the floor of the living space, and generating an alert to one or more authorized persons if the one or more body parts of the digital twin are identified as being within a defined distance of the floor.

The movement of the person may be monitored using at least one video camera.

The method may include storing video data of the at least one video camera for verification of a fall by authorized persons.

The method may further include obfuscating the video data to protect the privacy of the person in the living space.

The at least one video camera may monitor the person only when movement is detected and for a defined period following termination of movement.

### BRIEF DESCRIPTION OF THE DRAWINGS

FIG. 1 is a depiction of one embodiment of a system of the invention;

FIG. 2 is a flow chart defining the logic of one embodiment of an anomaly detection algorithm implemented in an AI system;

FIG. 3 is a flow chart defining the logic of one embodiment of an anomaly detection and corroboration algorithm implemented in an AI system;

FIG. 4 is a depiction of part of another embodiment of a system of the invention,

FIG. 5 is a depiction of yet another embodiment of a system of the invention,

FIG. 6 is a depiction of yet another embodiment of a system of the invention, and

FIG. 7 is a depiction of an embodiment of the control logic for identifying falls.

### DETAILED DESCRIPTION OF THE INVENTION

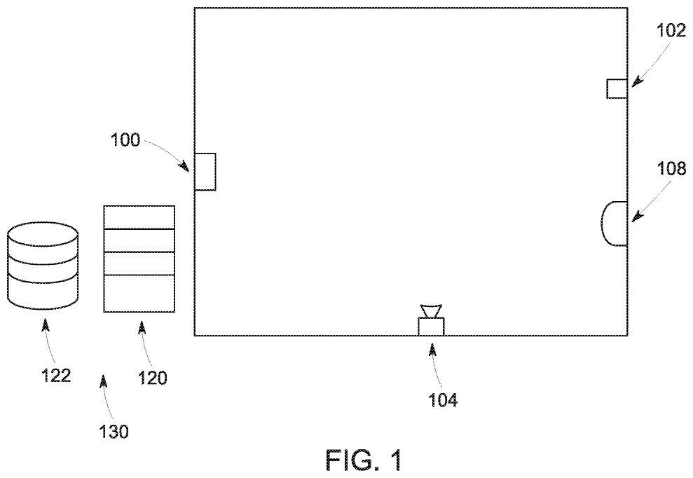

One embodiment of an interactive communication platform of the invention is shown in FIG. 1. It includes an image capture device 100 mounted on a wall in a user's apartment, a microphone 102 for capturing verbal and non-verbal sound information, and a speaker 104 for verbally addressing the user.

The image capture device 100, microphone 102, and speaker 104 are connected to a processor, which is not shown but is included in the housing for the speaker 104. The image capture device 100 and microphone 102 are communicatively connected to the processor by means of short-range wireless connections, in this case, Bluetooth.

The image capture device 100 and microphone 102 in this embodiment are implemented to always be on, so as to capture all activities of the user. Instead, the image capture device 100 (which in one implementation comprises an RGB video camera) may include a motion detector and may only be activated when movement is detected by the motion detector, or when the microphone 102 turns on. Similarly, the microphone 102 may only turn on when a sound is detected by the microphone or the image capture device 100 turns on.

Both the image capture device 100 and microphone 102 can therefore pick up emergencies such as the user falling. The image capture device 100 will detect when a user drops to the ground, either slowly or suddenly, while the microphone 102 will pick up thuds or percussion sounds associated with a fall, or verbal exclamations by a person, indicative of a fall, even if the user is outside the viewing field of the image capture device 100. In some instances the data from one device may identify an anomaly in the visual or sound data but be insufficient to clearly define the anomaly as a triggering event (also referred to herein as an emergency event, or flagging event). In such cases corroboration by a second device for the same or a related time-frame may serve to provide the necessary evidence to elevate an anomaly to an event, requiring third party intervention. For instance, both devices could pick up anomalies or flagging events such as a person falling, allowing the information of one device, e.g., the image capture device 100 to be corroborated against that of the other device (in this case the microphone).

The system of the present embodiment also includes a memory (not shown) connected to the processor and configured with machine-readable code defining an algorithm for analyzing the data from the image capture device 100 and microphone 102 and comparing it to a database of previously captured image data of people falling, and to previously captured sounds (both verbal exclamations and non-verbal sounds) associated with persons falling. In the case of verbal sounds, e.g. exclamations, the previously captured verbal data is preferably based on verbal data captured from the user, in order to ensure comparison with the user's actual voice, with its distinct frequency, timbre, and other vocal attributes. The database information may be stored in a section of the memory or a separate memory.

In this embodiment some of the processing is done locally, as it relates to the comparison of data from the image capture device 100 and microphone 102 to the previously captured data. The present embodiment also does some of the processing remotely by including a radio transceiver (not shown), which in this embodiment is implemented as a WiFi connection to a server 120

In one embodiment, the anomaly analysis is implemented in software and involves logic in the form of machine readable code defining an algorithm or implemented in an artificial intelligence (AI) system, which is stored on a local or remote memory (as discussed above), and which defines the logic used by a processor to perform the analysis and make assessments.

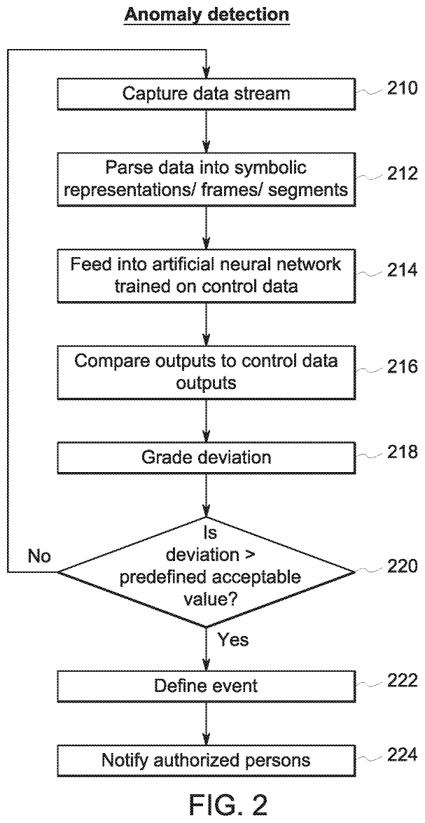

One such embodiment of the logic based on grading the level of the anomaly, is shown in FIG. 2, which defines the analysis based on sensor data that is evaluated by an Artificial Intelligence (AI) system, in this case an artificial neural network. Data from a sensor is captured (step 210) and is parsed into segments (also referred to as symbolic representations or frames) (step 212). The symbolic representations (also referred to as a digital twin) are fed into an artificial neural network (step 214), which has been trained based on control data (e.g. similar previous events involving the same party or parties or similar third-party events). The outputs from the AI are compared to outputs from the control data (step 216) and the degree of deviation is graded in step 218 by assigning a grading number to the degree of deviation. In step 220 a determination is made whether the deviation exceeds a predefined threshold, in which case the anomaly is registered as a flagging event (step 222) and one or more authorized persons is notified (step 224)

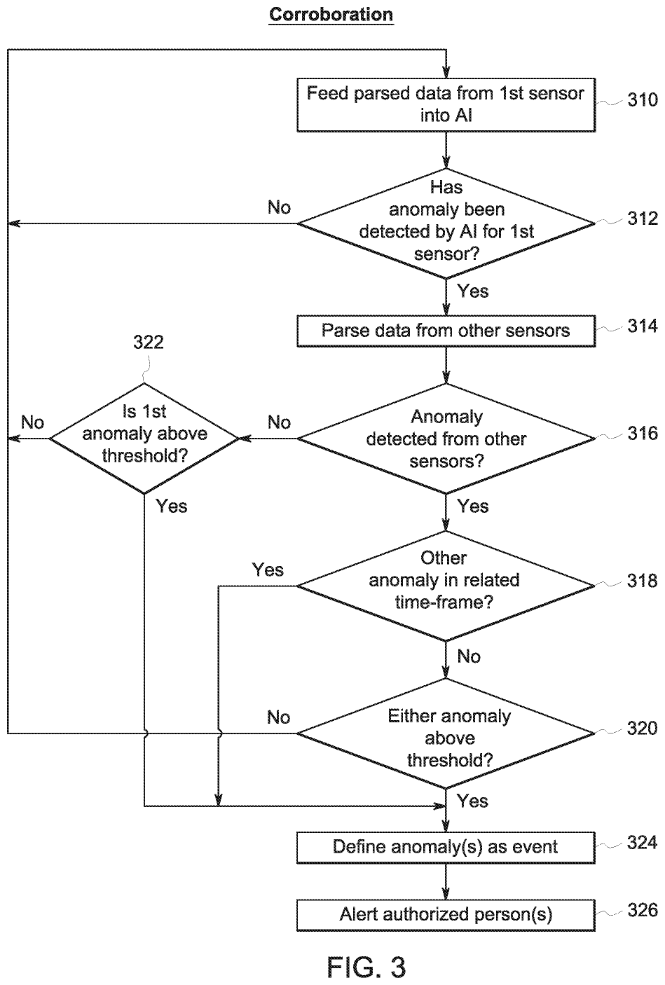

Another embodiment of the logic in making a determination, in this case, based on grading of an anomaly and/or corroboration between sensors is shown in FIG. 3.

Parsed data from a first sensor is fed into an AI system (step 310). Insofar as an anomaly is detected in the data (step 312), this is corroborated against data from at least one other sensor by parsing data from the other sensors that are involved in the particular implementation (step 314). In step 316 a decision is made whether any of the other sensor data shows up an anomaly, in which case it is compared on a time scale whether the second anomaly is in a related time frame (which could be the same time as the first sensor anomaly or be causally linked to activities flowing from the first sensor anomaly) (step 318). If the second sensor anomaly is above a certain threshold deviation (step 320) or, similarly, even if there is no other corroborating sensor data, if the anomaly from the first sensor data exceeds a threshold deviation (step 322), the anomaly captured from either of such devices triggers a flagging event (step 324), which alerts one or more authorized persons (step 326).

The server 120 also includes a database 122 for storing data from the image capture device 100 and microphone 102. In one embodiment it will capture and retain for a period of time, all of the data received from the image capture device 100 and microphone 102. In another embodiment it will only retain data associated with a flagging event identified by the local processor, where the local processor has determined that at least one of the two sensors (image capture device 100 or microphone 102) has picked up data that corresponds to previously captured data associated with a fall.

In order to protect the privacy of the person or people being monitored, the logic in the memory connected to the local processor may be configured to obfuscate the video data in order to create a blurred set of images of the retained image data. The logic may also use the parsed video data to generate avatars of the person or people being monitored. It will be appreciated that in a simplified embodiment, the system may avoid the need of the microphone, and rely purely on the video data to identify falls. In such an embodiment it is critical to protect the privacy of the people being monitored especially when monitoring the bathroom. In addition to obfuscating the image data, the logic in the memory may define blacked-out privacy regions, e.g., the toilet. Also, the logic may be configured to only capture video data when a flagging event is identified.

In the broader embodiment that includes a microphone, the database 122 also captures and retains sound files of non-verbal data associated with trigger events. In this embodiment the memory associated with the local processor includes logic for comparing non-verbal sounds received from the microphone to previously recorded non-verbal sounds, e.g. thuds corresponding to a person falling, thereby defining a falling event, which may trigger additional action, as is discussed further below.

In another embodiment, all of the processing is done remotely, in which case the database 122 includes previously captured image data, non-verbal sounds and voice-prints obtained from the user to define falling events.

Server 120 also includes a memory configured with machine readable code to define an artificial intelligence (AI) system (also referred to herein as an AI network), depicted by reference numeral 130. The AI system 130, inter alia, processes flagging events from one device (e.g. image capture device 100) and compares them with the other device (e.g., microphone 102) to corroborate a falling event for a corresponding time-frame.

As discussed above, the database 122, in one embodiment, includes previously captured image data, non-verbal sound data, and voice-prints, which allows the AI system to compare information captured by the image capture device 100 and microphone 102 to the previously captured data to identify falling events.

Certain potential falling events may require a closer view of the user, and the AI system is configured to identify the location of the user and zoom in on the user to capture a closer view of the person's body position or facial features. The facial expressions may provide information that the person is in pain or stress, based on comparisons to previously captured facial images of the user under different situations. The facial expression may include the configuration of the mouth, the creases formed around the mouth, creases along the forehead and around the eyes, the state of the eyes, and the dilation of the pupils.

The speaker 104 is integrated with the AI system to define a voice-bot for interacting with the user. It is configured to engage the user in conversation: in response to a fall event or potential fall event in order to gather additional information for purposes of corroborating or validating a fall event.

Upon the occurrence of a potential fall event, e.g. when the image capture device 100 or microphone 102 picks up data corresponding to a fall, data from the one device may be used to corroborate data from the other device. In the absence of sufficient information to warrant elevating the event to a trigger event that warrants third party intervention, the devices are configured to acquire additional information.

The image capture device, as discussed above, may zoom in on the user to assess body posture and facial features, and compare these to the image data in the database.

In response to image data suggesting a possible fall event, or in response to a verbal exclamation or non-verbal sound (e.g., one suggesting a falling event based on comparisons to previously captured sound files), the speaker 104 may engage the user in conversation, e.g., asking: “Are you alright?” or “Is everything alright?”.

Thus, in addition to the visual parameters (body posture and facial features captured by the image capture device 100), this allows a more detailed analysis of the speech-related parameters (as captured by the microphone 102).

In this embodiment, the voice signals are analyzed for intonation, modulation, voice patterns, volume, pitch, pauses, speed of speech, slurring, time between words, choice of words, and non-verbal utterances.

By analyzing the speech patterns of the verbal response or the lack of a response, the AI system may elevate a possible falling event to an emergency or trigger event, initiating a call to one or more persons in the database 122. For this purpose, the database 122 may include contact details of administrative staff responsible for the user, a physician or medical facility associated with the user, an emergency response entity, or a family member or emergency contact associated with the user, etc. The AI system, in this embodiment is configured to automatically contact one or more emergency numbers, depending on the situation, or connect the user with a contact person.

Also, the AI system may use the voice signals and images captured from a specific user, which are associated with one or more corroborated falling events, to refine the voice-prints and image data for the specific user. Thus, it becomes continuing teaching data for the AI system.

Similarly, the user may actively initiate a conversation or other interaction with the voice-bot by requesting an action (e.g. to connect the user by video or audio link with a specified person or number).

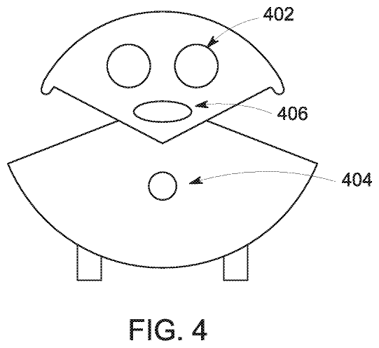

Another embodiment of the present system is shown in FIG. 4, wherein the AI system is implemented as a robot 400, which, in this embodiment is a mobile robot allowing it to approach the user for closer inspections, or to detect muffled or low-volume sounds, such as breathing or mumbling by the user.

This embodiment incorporates a image capture device 402, microphone 404, and speaker 406 in the robot 400. As in the FIG. 1 embodiment, the robot 400 may include both a processor for local processing of data captured by the image capture device 402 and microphone 404, as well as a transceiver (cell phone or internet-based radio transceiver) to communicate with a remote server such as the server 120 discussed above with respect to FIG. 1.

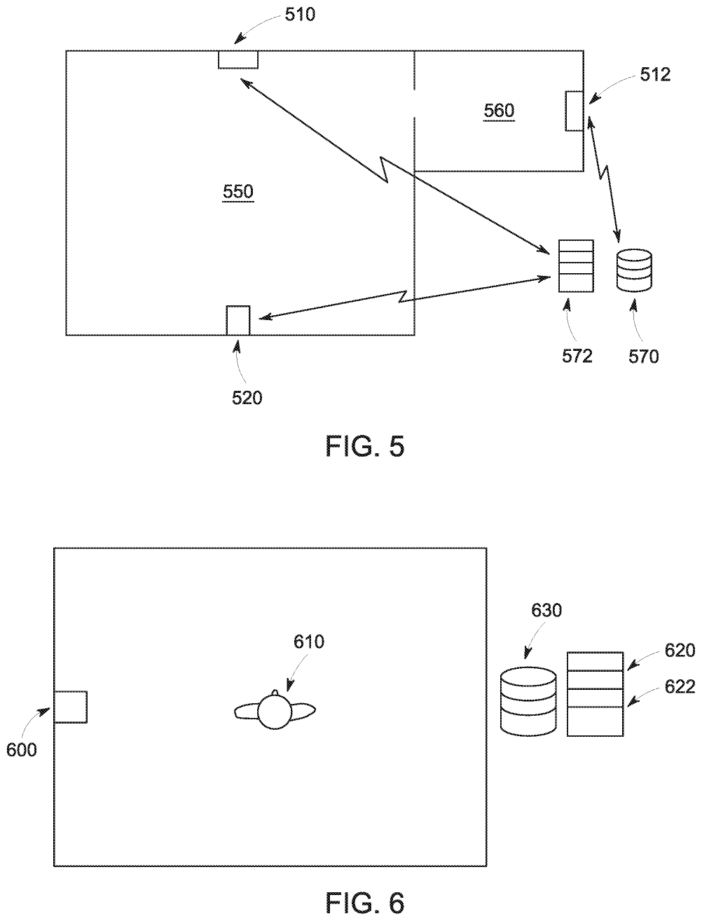

Another embodiment of a system of the present invention is shown in FIG. 5, which includes three image capture devices, comprising two radar devices 510, 512 and an infrared camera 520. The radar devices 510, 512 may be more prone to incorrectly trigger a fall alarm (also referred to as generating false positives) e.g., due to interference from metal objects or other blocking elements. The infrared camera 520 may therefore be triggered to turn on only if radar device 510 detects a fall, thereby allowing the infrared camera 520 to corroborate the fall detection by the radar device 510. In another configuration both the radar devices 510, 512 and the infrared camera 520 may capture images continuously. The benefit of the second configuration is that the devices can corroborate each other. Thus, for example the risk of failing to detect a fall (false negatives) can be minimized. In this embodiment, the infrared camera 520 is limited to the living and sleeping quarters 550 of the apartment. In order to protect the privacy of the user, the infrared camera 520 does not cover the bathroom 560. In order to supplement the radar data while a user is in the bathroom, an algorithm on a memory device 570 connected to a processor 572 (which in this embodiment is implemented as an edge server) starts a timer and generates an alert if the user's duration in the bathroom exceeds a predefined time, or exceeds the maximum duration previously recorded for that user in the bathroom, by a predefined time. In this way false alarms and undetected falls are minimized while still affording the user a high level of privacy.

Yet another embodiment of the invention is shown in FIG. 6, which includes one sensor in the form of a video camera. In this embodiment the system is implemented to monitor the falls of a person (also referred to as a patient or monitoree) in the patient's environment, e.g., the patient's apartment or home. In this embodiment, the system includes a camera 600 for capturing image data of the patient 610, and a processor 620 and control memory 622, which in this embodiment are defined by a server. The control memory 622 is configured with machine readable code to define a parsing algorithm for parsing the image data to identify previously defined body parts, generating a digital twin (e.g., an avatar) of the person with corresponding body parts, controlling the movement of the digital twin in relation to movement of the patient, and monitoring the movement of the digital twin by means of a comparison algorithm to pre-stored data in a data memory 630, to identify events, which in this embodiment are accurate enough to constitute flagging events. These flagging events may include falls by the patient, repetitive behavior or other activities that cause alarm or require intervention. One or more of the parsing algorithm, and comparing algorithm, may include an artificial intelligence (AI) system.

The digital twin may also be displayed on a user device, e.g., smart phone, tablet or laptop of a person taking care of the patient, to allow the user to verify flagging events. The user device is in communication with the server 620, 622 and data memory 630, e.g., by means of a web application on the server and HTTP or by configuring the user device with a native mobile app. As discussed in the previous embodiments, trigger events may also be corroborated by a second sensor such as a microphone or other monitoring device, however, the accuracy of the camera 600, coupled with the parsing algorithm typically is accurate enough on its own and simultaneously protects the privacy of the patient by making use of a digital twin. Insofar as video data is required to verify a fall, the image may be obfuscated, e.g., blurred to protect the privacy of the patient. Un-obfuscated images may be made available only to persons that have been authorized, e.g., the patient's physician.

The generating of the avatar and controlling the movements of the digital twin in relation to the movements of the patient may be implemented using pre-existing software such as wire-frame figures and other depictions (all referred to herein for simplicity, as avatars).

The processing of the image data from the camera 600 may be performed locally e.g., using an edge processing arrangement, or remotely at the server, which may comprise a dedicated server or a cloud server network. In the present embodiment the camera 600 was implemented as a video camera in the visual spectrum (RGB camera). However, it will be appreciated that other frequency bands, e.g., infra-red could be used. The benefit of the present embodiment, which generates and avata, is that it protects the privacy of the patient by depicting the patient as an avatar, thereby avoiding third parties seeing the actual patient during monitoring, e.g., when the patient may be in an unclothed or semi-clothed state.

In order to define the space within which the avatar operates, the avatar is spatially positioned within a virtual environment, also referred to as a doll house of the patient's apartment. This involves first capturing the spatial parameters of the environment, as is discussed in U.S. Pat. No. 10,049,500.

In the present embodiment, the monitoring of a patient in an apartment is described in order to monitor for things like falls, thereby providing a non-intrusive way of enhancing the safety of the patient, e.g., an elderly person in a continuous care retirement community (CCRC).

It will, however, be appreciated that the same approach can be used to monitor other persons such as inmates in a prison. While the issue of privacy may not be paramount in the case of a prison environment, the generation of an avatar for each inmate, provides for more reliable comparison to pre-defined permissible or impermissible acts or behavior that requires further investigation. These acts may include suspicious interactions, violent behavior between inmates, movement into areas that are off limits, etc.

The present embodiment has yet another advantage, since it provides a simple way of identifying between multiple patients or inmates (collectively monitorees) in a communal area. Since each monitoree is typically associated with a personal environment, each monitoree may be assigned an identity code based on their personal environment. This identity code may be implemented as a different color code associated with each avatar, or a visual tag that accompanies the avatar. The visual tag may, for example, include the person's name and other pertinent information, such as their personal environment, e.g., Ms Emma Smith, Room C128. This makes it easy to identify who is involved in interactions between monitorees and thereby help reduce unwanted behavior such as abuse of a patient by a nurse.

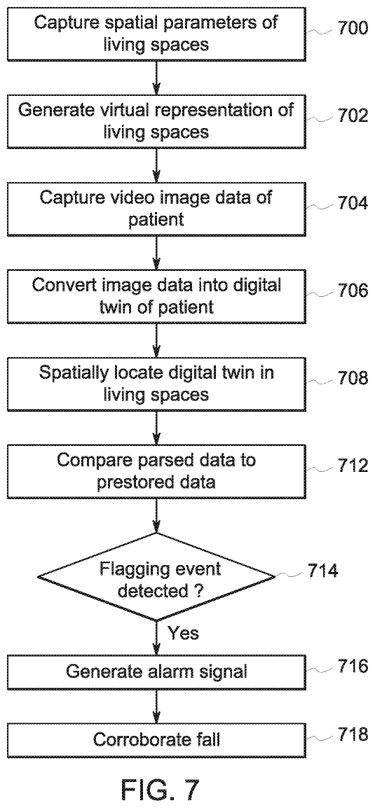

One embodiment of the comparison logic to identify a fall event of a patient in a CCRC is shown in FIG. 7. The spatial parameters of the patient's living spaces are captured in Step 700 in order to subsequently allow a representation of the patient to be spatially located in the patient's living spaces. The details of capturing the spatial parameters can be performed in a number of ways, known in the art, as discussed in U.S. Pat. No. 10,049,500. In one embodiment the x, y, z coordinates of each of the living spaces (e.g., living room, bedroom, kitchen, and bathroom) are defined with respect to a reference point. A virtual representation of the living spaces is then generated (Step 702) and made available as a virtual image (doll house) on the user's device.

Using a camera, such as the camera 600 discussed with respect to FIG. 6, image data of the patient is captured (Step 704). Using software, such as that provided by XNect, the image data of the patient is parsed to identify critical moving parts (in this embodiment, the head, spine, upper and lower arms, and upper and lower legs) and converted into a digital twin representation of the patient (Step 706). The digital twin is spatially located within living spaces (Step 708) as discussed in U.S. Pat. No. 10,049,500, which is incorporated herein by reference.

In Step 712, the relative positions of the moving parts and their location relative to the floor (z=0) are compared to pre-stored data in a data store (e.g. the database 630) which corresponds to body configurations of a person lying on the floor or slumped to the floor (also referred to herein as a fall configuration). If a fall configuration is detected (Step 714), the processor generates a flagging event (Step 716), which generates an alarm signal (FIG. 718) to allow the flagging event to be verified or corroborated by a person monitoring the digital twin on a user device, or by a second sensor (Step 720). Once a fall has been verified, help can quickly be sent to assist the patient.

While the present invention has been described with respect to specific embodiments, it will be appreciated that the invention could be implemented in different manners, with additional sensors and communication devices, and with differently configured processing of the data captured by the sensors, without departing from the scope of the invention.

What is claimed is:

1. A fall detector to detect falls by a person in a living space, comprising
an image capture device, and
a processor connected to a memory that includes machine-readable code defining an algorithm for analyzing data from the image capture device to detect falling events, wherein the image capture device is connected to a motion detector in order to capture only select information, and the algorithm includes logic to generate a digital twin of the person and monitor the distance of one or more body parts of the digital twin relative to the floor of the living space.

2. The fall detector of claim 1, wherein the algorithm includes logic to obfuscate at least part of the image.

3. The fall detector of claim 1, wherein the algorithm includes logic to define a flagging event if the distance of said one or more body parts of the digital twin relative to the floor drops below a defined value.

4. The fall detector of claim 3, wherein the algorithm is configured to generate an alert to one or more persons when a flagging event is detected.

5. A fall detector to detect falls by a person in a living space, comprising at least two image capture devices for detecting falls,
a processor,
a control memory connected to the processor and configured with machine-readable code defining an algorithm, and
a data memory, wherein the algorithm includes logic to corroborate a fall detected by one image capture device by comparing the data from the other image capture device for a related time frame.

6. A fall detector of claim 5, wherein the image capture devices operate in different frequency bands.

7. A fall detector of claim 6, wherein one image detector comprises a radar detector and a second image detector comprises an RGB or IR camera.

8. A fall detector of claim 7, wherein the radar detector is mounted in privacy-sensitive locations of the living space.

9. A fall detector of claim 8, wherein the algorithm includes logic to time the person in privacy-sensitive locations and identify anomalies in the timing and corroborate anomalies in the time spent by the person in the privacy-sensitive location with data from the radar detector for a related time frame.

10. A method for detecting a fall by a person in a living space, comprising
monitoring the person in the living space by generating a digital twin of the person,
monitoring one or more body parts of the digital twin relative to the floor of the living space, and
generating an alert to one or more persons if the one or more body parts of the digital twin are identified as being within a defined distance of the floor.

11. The method of claim 10, wherein the movement of the person is monitored using at least one video camera.

12. The method of claim 11, further comprising storing video data of the at least one video camera for verification of a fall by authorized persons.

13. The method of claim 11, further comprising obfuscating the video data to protect the privacy of the person in the living space.

14. The method of claim 11, wherein the at least one video camera monitors the person only when movement is detected and for a defined period following termination of movement.
